(19) United States
(12) Patent Application Publication
(10) Pub. No.: US 2003/0158140 A1
(43) Pub. Date: Aug. 21, 2003

(54) Splicing as target for identifying new active substances

(76) Inventors: Peter Schreier, Koln (DE); Christian Aichinger, Koln (DE)

Correspondence Address: BAYER CROPSCIENCE LP, 100 BAYER ROAD, PITTSBURGH, PA, 15205, US

(21) Appl. No.: 10/265,866

(22) Filed: Oct. 8, 2002

(30) Foreign Application Priority Data
Oct. 15, 2001 (DE) 101 50 783.6

(51) Int. Cl. A01N 25/00; A01N 43/04; C12Q 1/68; C12N 1/18; C12N 15/74; C12N 5/06

(52) U.S. Cl. 514/44; 435/6; 435/254.21; 435/348; 435/320.1; 504/116.1

(57) ABSTRACT

The present invention relates to new deoxyribonucleic acid (DNA) constructs, to vectors and host organisms which contain these DNA constructs and which, owing to their use in specific methods, are suitable for indicating the loss of correct splicing, and to the use of these transgenic organisms or cells for identifying new active substances, and, finally, to methods of finding new active substances which are capable of preventing correct splicing and to the use of these methods in high-throughput screening (HTS) or ultra-high-throughput screening (UHTS).

The present invention relates to new deoxyribonucleic acid (DNA) constructs, to vectors and host organisms which contain these DNA constructs and which, owing to their use in specific methods, are suitable for indicating the loss of correct splicing, and to the use of these transgenic organisms or cells for identifying new active substances, and, finally, to methods of finding new active substances which are capable of preventing correct splicing and to the use of these methods in high-throughput screening (HTS) or ultra-high-throughput screening (UHTS).

Over 20 years ago, interrupted genes and splicing of the pre-mRNA (precursor messenger ribonucleic acid) was discovered in eukaryotes and their viruses (cf. Berget et al. (1977), Sambrook (1977)). During splicing, what are known as introns (i.e. non coding DNA regions within a sequence encoding a protein) are excised from the primary gene transcript with the precision of a single nucleotide. In doing so, what are known as exons (i.e. the coding DNA regions) are combined and result in mRNAs which can be translated correctly. At the beginning, within and at the end of an intron, the sequence of the primary transcript contains conserved sequences which are among the decisive splicing factors. A conserved sequence 5′-GU . . . AG-3′ is located at the very ends. An adenosine unit is located in general 10 to 40 nucleotides upstream of the 3′ splicing site and acts as branching site. Chemically, two trans-esterification reactions proceed sequentially during splicing (see FIG. 1). The first step is the cleavage of the border between the 5′ exon and the 3′ residue of the pre-mRNA. Here, the 2′-OH group of the invariant adenosine unit carries out a nucleophilic attack on the 5′-phosphate group of the guanosine unit of the intron, giving rise to what is known as the Lariat structure (see FIG. 1A). Then, in the second step, the free 3′-OH group of the exon which has cleaved off during the first reaction attacks the phosphodiester bond at the 3′ splicing site (see FIG. 1B). As yet, no definitive studies have been carried out into the extent to which splicing differs between different organisms.

The fact that the splicing apparatus (spliceosome) is at least as complicated in structure as a ribosome was first described in 1985 (Grabowski et al. (1985)). The spliceosome consists of a number of snRNAs (small nuclear RNAs) and a series of proteins. The number varies depending on the organism. In yeast, for example, 5 snRNAs and approximately 50 proteins are involved. In addition, approximately 100 further proteins are involved in the splicing process. The spliceosome is responsible for more than 10 RNA-RNA interactions in the correct sequence and the correct timing of their redissolution, so that spliceosomes, like ribosomes, are highly complex ribonucleoprotein (RNP) machines. The spliceosomes must be newly assembled for each excision of an intron (what is known as an assembly). The assembly of a spliceosome is a rigidly structured dynamic sequence of individual cuts, which comprises the hydrolysis of a multiplicity of ATP molecules and the structural reorganisation of a number of proteins and RNA molecules. The precision and sequence over time of this procedure, in turn, is governed by various other proteins.

Eukaryotes contain a large number of genes which must be spliced in order to arrive at the corresponding correct mRNAs and thus functional proteins. As a rule, interference with the assembly of the spliceosome and with the splicing leads to cell death.

New active substances which meet the increasing requirements regarding efficacy, ecofriendliness, resistance behaviour and costs are constantly sought in a number of fields in crop protection and in medical applications. For example, the undesired growth of fungi and weeds or attack by pests result every year in substantial damage in agriculture. These eukaryotes, with their undesired growth, can be controlled for example by fungicides, herbicides or insecticides. There is therefore a constant demand for new substances or classes of substances which can be developed into potent and ecofriendly new active substance preparations. In the case of fungicides, it is generally customary to search for such new leaf structures in greenhouse tests. However, such tests are labour-intensive and expensive. Accordingly, the number of substances which can be tested in the greenhouse is limited. An alternative to such tests is the use of what are known as high-throughput methods (HTS=high-throughput screening) or ultra-high-throughput methods (UHTS=ultra-high-throughput screening). Here, a large number of individual substances is tested in an automated method with regard to their effect on single cells, individual gene products or genes. If an effect is found for certain substances, these substances can be studied in conventional screening methods and, if appropriate, developed further.

Advantageous targets for active substances, in particular for fungicides, herbicides, insecticides or pharmaceuticals, are frequently searched for in essential biosynthesis pathways. For example, an ideal fungicide is a substance which inhibits a gene product playing a decisive role in the manifestation of pathogenicity of various fungi.

In the medical-pharmaceutical field, it is known that incorrect splicing can lead to a variety of clinical pictures. Incorrect splicing may cause decisive enzymes to be produced in an inactive form (Zanelli et al. (1990)). Other studies suggest that a defective gene product leads to considerable interference with the formation of snRNPs, thus inhibiting the splicing apparatus (Fischer et al. (1997)). It appears that certain alternative splicing variants, inter alia, play a decisive role in the metastatic spread of cancer cells (Sherman et al. (1996)).

WO 00/52201 discloses an in-vitro assay system for recognizing a splicing reaction. The disadvantage of this assay system is the relatively complicated preparation of nuclear extracts of the cells used and the preparation and immobilization of suitable splicing constructs, and the detection of the splicing products formed.

WO 00/67580 discloses a further in-vitro method for identifying compounds which have an effect on eukaryotic splicing. Again, the necessity of preparing cell extracts for use in a splicing assay is disadvantageous. A further disadvantage is that the detection of whether splicing has taken place or not is carried out by gelelectrophoretic methods.

The present invention is based on the approach of using the spliceosome as target in the search for new active substances in such a way that a suitable in-vivo method can be used for detecting whether its function is adversely affected, i.e. reduced in terms of activity, or fully suppressed.

In the present context, spliceosome is understood as meaning the entire complex required for splicing. Essentially, it is composed of a variety of snRNAs which, as a rule, are present incorporated in ribonucleoprotein particles (known as snRNPs) and a series of further splicing factors, among them RNA-binding proteins, which support the splicing process.

Thus, the spliceosome offers a large number of targets for a wide range of active substances. Highly suitable as targets are not only the abovementioned components of which the spliceosome consists, but also those regulators which are directly and/or indirectly involved in the correct assembly of the spliceosome or one of its units, and all those factors which influence correct expression of the proteins involved in terms of timing and/or location, or which affect the assembly of the spliceosome.

The invention therefore relates to a method of detecting the functionality of the splicing process as such, in particular in vivo.

For example, the functionality can be detected in such a way that DNA constructs are first prepared which, in addition to a DNA sequence containing all the necessary elements for successful splicing (for example an intact intron or a corresponding sequence), have a reporter gene. When these DNA constructs are subsequently introduced by means of transformation, i.e. either with the aid of a vector or by transformation with linear DNA, into a host organism or a host cell capable of splicing, the reporter gene is translated either when splicing is intact or when splicing is adversely affected, preferably when splicing is adversely affected, depending on the position of the reporter gene within the DNA construct, and the functionality of the splicing process is thus indicated.

If, for example, a cell is modified with such a DNA construct in such a manner that the reporter gene or a necessary component of the reporter gene is spliced out under natural conditions, that, in the event of splicing being inhibited, is transcribed and, as a consequence, translated, it is possible to differentiate between the individual cases as follows:

| | Splicing | Reporter gene |
|---|---|---|
| without inhibitor | yes | not active |
| with inhibitor | no | active |

Reporter gene is understood as meaning, in the present context, a coding sequence with a readily detectable gene product or its activity, which reporter gene can be linked to a suitable promoter. The gene product of the reporter gene must be characterized biologically well enough for an active centre, or a protein domain required for function, to be known (functional unit). Examples of reporter genes which may be mentioned are the following: the Escherichia coli lacZ gene, which encodes β-galactosidase; various luciferase genes, i.e. enzymes which catalyse reactions which lead to bioluminescence; the GFP (green fluorescent protein) gene or its variants from the pacific jellyfish species Aequorea victoria, whose translation gives rise to a fluorescent protein as product.

Transformation for the purposes of the invention is understood as meaning a general method of introducing DNA into higher cells. Either vectors can be used for this purpose, or transformation is effected with linear DNA. In general, known selection markers such as, for example, hph (hygromycin phosphotransferase gene leads to hygromycin B resistance), npt II (neomycin phosphotransferase gene leads to kanamycin resistance), NAT (leads to resistance to CLONAT, Hans-Knöll-Institut, Jena), cbx (the gene for the succinate-dehydrogenase iron-sulphur subunit leads to resistance to carboxin), pyr6 [orotidine 5′-monophosphate decarboxylase gene, complementation of ura⁻ (Δpyr6) strains], which permit identification of those cells into which foreign DNA has successfully been introduced, are used for verifying the success of the transformation.

Vectors which can be used are all those viral vectors, plasmids, phasmids, cosmids, YACs, BACs, artificial chromosomes or DNA-coated particles suitable for particle bombardment which are used in molecular-biological laboratories.

The term host cells and host organisms as used in the present context refers to cells or organisms which do not naturally contain the DNA constructs according to the invention. If the term is intended to encompass both possibilities, host cells and host organisms are linguistically combined by the term host.

Suitable host cells or host organisms are eukaryotic cells, such as fungal cells, insect cells, plant cells, frog oocyte cells and cells from mammalian cell lines, or else intact organisms such as Volvox spheroids, Drosophila embryos or Daphnia larvae. Single cells capable of being transformed are preferably used, especially preferably fungal cells, very especially preferably cells of Saccharomyces cerevisiae, Magnaporthe grisea, Aspergillus nidulans, Cochliobulus heterostrophus, Nectria hematococca, Botrytis cinerea, Gaeumannomyces sp., Pichia pastoris and Ustilago maydis, very especially preferably cells of Ustilago maydis.

The invention therefore relates to a method of detecting the functionality of the splicing process, which is characterized in that host cells or host organisms containing the DNA constructs according to the invention are examined for the activity of the reporter gene. In this context, it does not matter whether a measurable signal is obtained upon correct splicing or upon lost or reduced activity of the splicing.

Thus, the invention relates to a method of detecting the functionality of the splicing process, which is characterized in that

(A) a DNA construct is prepared whose existence in a eukaryotic organism leads to the possibility of detecting the loss of correct splicing;

(B) the DNA construct of step (A) is introduced into a host cell or a host organism;

(C) the presence or absence of the reporter gene product is verified in order to detect the splicing activity.

The DNA constructs which are generated in step (A) must have several characteristics to have available a detectable result upon the loss of correct splicing. The individual components of these constructs are a promoter which is active in eukaryotes, an intron sequence, i.e. a DNA sequence, which has all the functional elements of an intron, and a reporter gene, all these components being operably linked, independently of their sequential arrangement.

In this context, operably linked means, for the purposes of the invention, that the formation of the reporter gene product is made possible either upon intact or upon adversely affected/inhibited or upon fully suppressed splicing.

The DNA constructs thus provide reporters which no longer have a biological activity either during the functional splicing process or during the adversely affected or fully suppressed splicing process. Thus, the presence or absence of the activity can be detected in all cases. Preferably used DNA constructs are those whose reporter produces a detectable signal during an adversely affected or eliminated splicing process. In this context, it does not matter whether assembly of the spliceosome is prevented in the first place, or only the splicing process itself.

Suitable promoters are all those eukaryotic promoters which make possible the transcription of the reporter gene. Promoters which can be used are constitutive, regulable or synthetic promoters which, in the assay system in question, transcribe the DNA construct comprising the reporter gene.

In an especially preferred embodiment, Ustilago maydis promoters, very especially preferably the regulable crg1 promoter (Bottin et al. (1995)), the constitutively active hsp70 promoter (Holden et al. (1989)) or the synthetic otef promoter (Spellig et al. (1996)) or the synthetic oma promoter (whose sequence corresponds to SEQ ID NO. 14) are employed.

Constitutive promoters are those promoters which make possible the continuous transcription of RNA with a low level of regulation.

Regulable promoters are those promoters whose activity can be controlled by specific factors in such a way that the transcription rate can be increased or reduced.

Synthetic promoters are those promoters which do not occur naturally since they are composed of various promoters or promoter fragments or their regulatory elements. They may comprise the characteristics of constitutive and of regulable promoters.

The DNA construct according to the invention furthermore contains an intron sequence. For the purposes of the present invention, this is understood as meaning a DNA sequence which has all the features of an intron, is recognised as intron by the spliceosome, and is excised from the DNA when splicing is intact.

The recognition features required include a 5′ splicing site starting with the nucleotides 5′-GU-3′, preferably starting with 5′-GUAAGU-3′. Moreover, a 3′ splicing site starting with the nucleotides 5′-AG-3′, preferably with 5′-YAG-3′, where Y is a pyrimidine base (thymidine or cytosine), is required.

In addition, what is known as a Lariat binding site may optionally be present upstream of the 3′ and of the intron sequence in the form of an adenosine nucleotide (as is the case in yeasts, for example Saccharomyces cerevisiae). As a rule, an adenosine unit is indeed present as branching site at a distance of, in general, 10 to 40 nucleotides (in yeasts 14 to 18 nucleotides) upstream of the 3′ splicing site. According to present knowledge, this region is not precisely defined and, depending on the organism, may vary within a substantial range which even exceeds the limits of the abovementioned range. This binding site is not absolutely required for carrying out the method according to the invention.

Intron sequences which are preferably employed are those which contain no start and/or stop codons so that the translation of the entire DNA construct is not adversely affected by these codons.

Also encompassed in accordance with the invention are those intron sequences which have been modified accordingly to satisfy the abovementioned requirements. This particularly applies to modifications in which any start and stop codons which prematurely initiate or terminate translation are removed. Also of interest in accordance with the invention are variations of the 5′ or 3′ splicing sites which bring about an improved splicing efficacy (for example by improving binding of the spliceosome to the pre-mRNA).

A modified intron is still understood as meaning, for the purposes of the invention, an intron which, following a suitable modification which may also take the form of a mutation, for example a point mutation, retains its function as an intron, that is to say is recognised as such by the spliceosome and spliced out. In the present context, such an intron is also termed a functional intron.

In contrast, a mutated intron is understood as meaning, for the purposes of the invention, an intron which, following modification, has lost its function as an intron (functionless intron), i.e. which is no longer recognised by the spliceosome and thus not spliced out.

Likewise encompassed in accordance with the invention are those intron sequences which have been put together on the basis of various known sequences and which have retained the characteristics of an intron. This particularly applies to introns whose consensus sequences were derived from the analysis of the Ustilago maydis genome. Known introns which can be used in accordance with the invention are, for example from U. maydis, the four introns of the lga2 gene or the three introns from the pra1 gene (Urban et al. (1996), Bölker et al. (1992)).

The intron sequence of the modified intron as shown in SEQ ID NO. 10 is preferably used.

Suitable reporter genes are: GFP and variants or derivatives (for example eGFP, yGFP, cGFP), lacZ, LUX, GUS, CAT, orotidine 5′-monophosphate carboxylase, nitrate reductase.

The structure of the DNA constructs according to the invention, i.e. the sequence in which the individual components are arranged, depends on the units used (promoter, intron sequence, reporter gene). Thus, it is generally possible to arrange the intron sequence upstream of the reporter gene or within the reporter gene. Preferably, the intron sequence followed by the reporter gene is arranged downstream of the promoter. However, it is also possible to prepare the DNA construct such that the intron sequence overlaps with the reporter gene. In a very especially preferred embodiment, the transition from the intron sequence to the reporter gene is designed such that the start codon for the reporter gene starts 6 base pairs before the functional unit of this gene and that the subsequent reporter gene can thus contain an additional amino acid.

The units used in the DNA constructs according to the invention (promoter, intron sequence, reporter gene) are operably linked, independently of their sequential arrangement.

The invention therefore furthermore relates to DNA constructs whose existence in a eukaryotic organism leads to the possibility of detecting the loss of correct splicing.

The invention preferably relates to DNA constructs consisting of a promoter which is active in eukaryotes, a DNA sequence which has all of the abovementioned functional elements of an intron, and a reporter gene.

FIG. 2 is a schematic representation of a possible structure of a DNA construct according to the invention. FIG. 2A shows an example of a general structure of such a DNA construct consisting of promoter (P), intron sequence (I) and reporter gene (R).

In a preferred embodiment (cf. FIG. 2B), the expression of the DNA construct used is conferred by the strong otef promoter (identified in FIG. 2B as Potef) (Spellig et al. (1996)). The oma promoter can be used as an alternative (SEQ ID NO. 14). The modified endogenous intron no. 1 from the U. maydis lga2 gene (Urban et al. (1996), SEQ ID NO. 10) is used as test intron in the DNA construct. Here, position 3 of the 5′ splicing site is modified (G is exchanged for A) and thus adapted to the predominant consensus. In this case, it has the sequence 5′-GTAAGT-3′. The 3′ splicing site has the sequence CAG (encoding the amino acid glutamine (Q)), while the start codon AUG (encoding the amino acid methionine (M)) is located directly upstream. This arrangement results in an artificial intron which has neither a start codon nor a stop codon in the reading frame in question. The eGFP allele (Clontech) acts as reporter gene.

After introduction into a host cell or host organism for use in an assay, the structure of the DNA constructs permits a distinction to be made between cells in which splicing takes place and those in which the splicing function is inhibited. The results of the individual constructs at mRNA level are shown in FIG. 3. Under growth conditions (for example in standard nutrient media such as PD medium, PD=potato dextrose, or in suitable minimal media), splicing is not inhibited and intron sequences are removed. This leads to the start codon (AUG), which is present in the DNA construct, is spliced out. The transcribed GFT-mRNA contains no correct translation initiation signal, and no reporter gene activity can thus be detected (see FIG. 3A).

However, if an inhibitor prevents the splicing process, the intron, and thus also the AUG start codon, are retained (see FIG. 3B). The translation can start at the start codon of the GFP gene, and the GFP protein is expressed.

Since an inhibitor for mRNA splicing has not been available to date, a plasmid was prepared to simulate this situation (positive control) by mutating the 5′ splicing site at one base (see FIG. 3C). This makes possible the expression of GFP even when no inhibition takes place since the start codon can now no longer be spliced out. Moreover, this construct can be used for determining the maximum of GFP fluorescence which is possible in the case of inhibition.

The individual cases (corresponding to FIG. 3) of this especially preferred embodiment can be compiled as follows:

| | | Splicing | GFP fluorescence |
|---|---|---|---|
| A | Functional intron without inhibitor | yes | no |
| B | Functional intron with inhibitor | no | yes |
| C | functionless intron without inhibitor | no | yes |

The present invention furthermore relates to a method of generating the DNA constructs according to the invention, which is characterized in that,

a) in a first step,

i) a suitable intron is amplified from a suitable gene of the genomic DNA of a suitable host cell or a suitable host organism with the aid of the primers I-5′ (for the 5′ flank of the intron) and I-3′ (for the 3′ flank of the intron), the sequence of the intron optionally being modified specifically by selecting a suitable primer,

ii) a suitable reporter gene is amplified from a suitable source, for example a plasmid, using the primers RG-5′ and RG-3′ (for the two flanks of the reporter gene),

b) in a second step, the two amplificates of step (a) independently of one another are cloned into a suitable plasmid I, giving rise to the two plasmids II (intron) and III (reporter gene),

c) in a third step,

i) the intron fragment is excised from plasmid II (intron) using restriction enzymes which generate the cleavage sites R1 and R2,

ii) the reporter gene fragment is excised from plasmid III (reporter gene) using restriction enzymes which generate the cleavage sites R2 and R3,

iii) a suitable vector containing a suitable promoter, for example a plasmid IV which has at least two different restriction cleavage sites R1 and R3, is restricted enzymatically in such a way as to give rise to the cleavage sites R1 and R3, and

iv) the three resulting fragments are ligated in such a manner that a plasmid V is obtained in which an intron sequence and the reporter gene are operably linked.

Suitable introns, host cells or host organisms and reporter genes which are required in step (a) of the method according to the invention for generating the DNA constructs have already been described above in connection with the method according to the invention for detecting the functionality of the splicing process.

Introns and reporter genes whose DNA sequences are already known or which have been made accessible by sequencing are preferably employed so that suitable primers for the amplification can be prepared.

Genomic DNA can be isolated by standard methods of molecular biology. For the isolation of the genomic DNA from U. maydis see Hoffmann and Winston (1987). If appropriate, the sequence of the intron is modified in step (a i). For example, the 5′ splicing site can be adapted to the prevailing consensus sequence, which is important for the splicing, by mutating the third nucleotide from G (guanine) to A (adenine). This point mutation can be introduced during the PCR reaction in a manner with which the skilled worker is familiar using suitable, for example, synthetic, primers. This gives rise to a modified, functional intron.

A suitable plasmid I which is used in step (b) is characterized in that it has different cleavage sites for at least three restriction enzymes, by means of which at least three different restriction cleavage sites R1, R2 and R3 can be generated. A suitable plasmid I which may be mentioned is, for example, plasmid pCRIITopo (Invitrogen). Cloning into this plasmid is carried out in such a manner that plasmid II, which subsequently contains the intron sequence, can be cleaved with a different combination of restriction enzymes than plasmid III, which contains the reporter gene, the cleavage sites being chosen so that the two fragments are identical at one end and can be ligated in step (c).

In step (c), fragments are first excised from three different plasmids in such a manner that in each case two ends can be ligated.

The vector used in step (c iii) (or plasmid IV) is chosen or constructed in such a way that it already contains a suitable promoter (for example pCA123).

The method according to the invention for generating the DNA constructs can not only be used for generating suitable constructs for the method for detecting the functionality of the splicing process, but also for generating constructs with specific properties (for example with a mutation in the 5′ splicing site of the intron sequence, cf. above and FIG. 3C).

Finally, plasmid V, which is obtained at the end of step (c) can be used to introduce the DNA construct according to the invention into a host cell or a host organism (step (B) of the method according to the invention).

In a preferred embodiment, for example the procedure hereinbelow is followed when generating a DNA construct:

a) In a first step,

i) for example the first intron of the lga2 gene is amplified from the genomic DNA of the U. maydis strain Um518 with the aid of the primers I-5′ (for the 5′ flank of the intron, for example the primer lga25′, SEQ ID NO. 1, by which the 5′ splicing site in the lga2 intron is mutated from G to A in comparison with the wild-type sequence) and I-3′ (for the 3′ flank of the intron, for example the primer CA52, SEQ ID NO. 2), and

ii) for example the egfp gene is amplified as the reporter gene from plasmid pCA123 (consisting of the otef promoter, the egfp gene, a pSP72 backbone and the cbx resistance) using the primers RG-5′ (for example the primer CA53, SEQ ID NO. 3) and RG-3′ (for example the primer 3′GFP-Not, SEQ ID NO. 4).

b) In a second step, the two amplificates of step (a) are cloned separately from one another into a suitable plasmid I (for example plasmid pCRIITopo from Invitrogen). Following this step, firstly plasmid II (for example plasmid pCRIITopo-lga2), which contains the intron sequence, and, secondly, plasmid III (for example plasmid pCRIITopo-UeGFP), which contains the reporter gene, are obtained.

c) In a third step,

i) the lga2 intron is excised from plasmid II (pCRIITopo-lga2) as a 74 bp BglII/SphI fragment,

ii) the egfp gene is excised from plasmid III (pCRIITopo-UeGFP) as a 726 bp SphI/NotI fragment,

iii) the vector (for example pCA123, containing the otef promoter) is restricted with the restriction enzymes BamHI and NotI, and

iv) the three resulting fragments are ligated together, giving rise to a plasmid V (p123-lga2-eGFP).

To obtain the DNA construct which is mutated at the 5′ splicing site of the intron sequence in such a manner that splicing can no longer take place, the primer lga25′mut (SEQ ID NO. 5), is used in step (a) in a further preferred embodiment, finally giving rise to plasmid VI (p123-lga25′mut-eGFP).

In step (B) of the method according to the invention, the DNA constructs according to the invention are introduced into a host cell or a host organism.

Accordingly, the invention also relates to host cells and host organisms containing the DNA constructs according to the invention.

Suitable host cells and host organisms have already been mentioned above in connection with the general description of the method according to the invention.

In step (B) of the method according to the invention, the DNA constructs according to the invention are introduced into a host cell or a host organism. General transformation methods which can be used for this purpose have already been described above.

In a preferred embodiment, intact organisms, especially preferably of U. maydis, very especially preferably the U. maydis strain Um518, are used.

When carrying out step (B) of the method according to the invention, a general procedure for introducing the DNA constructs into a host is followed in which the DNA constructs are optionally linearized with a suitable restriction enzyme and subsequently introduced into the host.

Restriction enzymes which are preferably used for linearizing the DNA are those which preferentially bring about the integration of the constructs at particular loci of the host organism.

In a preferred embodiment, for example, a procedure is followed in which plasmid V (p123-lga2-eGFP) is linearized with the restriction enzyme SspI and transformed into the genome of the haploid U. maydis strain Um518 by the PEG/protoplast method (cf. Schulz et al. (1990)). Cleavage with SspI preferentially integrates the constructs at the cbx locus of U. maydis. Here, the restriction enzyme SspI cleaves the cbx resistance gene in the open reading frame. The resistance to carboxin is conferred by a point mutation in the iron-sulphur subunit of the endogenous succinate dehydrogenase ip^{s}. The construct is now integrated, by homologous recombination, in such a way that it is flanked at the one side by the wt copy and at the other side by the resistance-conferring gene. Ectopic integrations are possible when, for example, the construct recircularizes during the transformation process or when parts integrate which have not been cleaved completely. The same procedure is followed with plasmid VI (p123-lga25′mut-eGFP).

To determine the site of integration of the splicing constructs, genomic DNA of the host is first isolated (cf. Hoffmann and Winston (1987)) and subsequently digested with specific restriction enzymes (for example HindIII and BamHI). For the detection, a PCR fragment from plasmid pCBX122 (Keon et al. (1991)) is used as probe in the case of integration into the cbx locus in Ustilago maydis.

The fact that the constructs are always integrated at the same gene locus makes possible a comparable expression in the different strains. The individual integration events are confirmed by means of PCR and by Southern blot analysis (see FIG. 4). Strains DS#873 and DS#877 (lane 4 and lane 8, respectively; FIG. 4) bear in each case two copies of the constructs p123-lga2-eGFP and p123-lga25′mut-eGFP.

The transformation success is verified after growing the colonies (for example of U. maydis on PD plates) by excitation of the eGFP fluorescence by a powerful light source with light of wavelength 485 nm (bandwidth 10 nm). The fluorescence of the eGFP protein is subsequently visualized by applying a filter with a transmissivity at 510 nm (bandwidth 10 nm).

The result is that all five transformants which bore the construct with a 5′-mutated splicing site emitted green fluorescence. The 5′ splicing site of the lga2 intron is modified by a point mutation using the primers CA52 (SEQ ID NO. 2) and lga25′mut (SEQ ID NO. 5) as described above, giving rise to an intron with the sequence shown in SEQ ID NO. 11. Strain DS#877 contains a corresponding construct. Accordingly, this point mutation in the 5′ splicing site suffices to prevent splicing of the intron. In contrast, GFP fluorescence was detected in none of the strains bearing a wt-lga2 intron. The modified wt intron (cf. the sequence of SEQ ID NO. 10) allows splicing, and, accordingly, no GFP fluorescence can be detected.

A further possibility of detecting splicing is at the molecular level by applying RT-PCR experiments (Reverse Transcription Polymerase Chain Reaction).

In general, a procedure is followed in these RT-PCR experiments in which total RNA is first isolated (cf Schmitt et al. 1990)). Then, polyA⁺ RNA is prepared therefrom and amplified by means of RT-PCR. To determine the exact length of the individual PCR products, the latter are sequenced. Whether the primary gene product of the reporter gene is present (in each case primer for the 5′ and 3′ end of this gene) which contains the intron (primer for the 5′ end of the intron and 3′ end of the reporter gene) can be determined directly by selecting suitable primer combinations. A third primer combination (primer for the 5′-UTR region and the 3′ end of the reporter gene) can be used for comparative purposes to distinguish between spliced and unspliced RNA since fragments of different lengths result.

In a preferred embodiment, a procedure is followed in which the U. maydis strains DS#873 and DS#877 are employed in RT-PCR experiments for detecting the GFP-mRNA. The U. maydis strain UMA3 into whose cbx locus the vector pCA123, which bears the eGFP gene under the control of the otef promoter, had been integrated and which thus expresses the egfp gene constitutively, acts as positive control for the RT-PCR.

To detect GFP expression independently of the mRNA species, a cDNA with the primers 5′GFP (SEQ ID NO. 6) and 3′GFP (SEQ ID NO. 7) was first selected. If a corresponding GFP-mRNA is present, a 680 bp fragment can be amplified. It emerged that only this fragment could be amplified in all of the test strains (gap size marker, upper third, lanes 1 to 3), that is to say that a GFP transcript was present.

To identify strains in which the intron was not spliced, a primer combination (intron/3′GFP, SEQ ID NO. 9/SEQ ID NO. 7) was selected which only gives a result when the intron is present. Here, only strain DS#877 gave a positive result in the form of a 746 bp mRNA fragment since this strain bears the 5′-mutated intron (cf. gap size marker, middle third, lane 2).

To differentiate directly between spliced and unspliced mRNA, the primer combination 5′UTR/3′GFP (SEQ ID NO. 8/SEQ ID NO. 7) is used. Depending on the splicing, amplicons of different lengths are generated. If splicing takes place, the GFP-mRNA is 734 bp in length; if no splicing takes place, however, it is 811 bp in length since the intron is still present. The analysis revealed a PCR fragment only 734 bp in length for the strains UMA3 and DS#873 (Lane 4: 1 kb+ marker, lower third, lanes 3 and 1, respectively) and an 811 bp amplicon for strain DS#877 (gap size marker, lane 2). This demonstrates that the difference with regard to transcript lengths can be attributed to splicing.

The result of these studies is shown schematically in FIG. 5B (splicing takes place) and FIG. 5C (no splicing). This result can be applied readily to the case of other reporter genes, introns and promoters.

The present invention also relates to methods of finding chemical compounds which act on the spliceosome and/or one of its components in a manner which leads to modulation of the splicing process, preferably to inhibition.

The present invention also relates to methods of finding chemical compounds which act on the assembly of the spliceosome and/or one of the components participating therein in a manner which leads to the modulation of the splicing process, preferably to inhibition.

The present invention therefore relates to a method of identifying inhibitors of the splicing process, which is characterized in that

(a) a DNA construct according to the invention (see above) is generated;

(b) this DNA construct of step (a) is introduced into a host cell or a host organism according to the invention (see above);

(c) the host cell or the host organism of step (b) is brought into contact with an individual substance or a mixture of a plurality of chemicals,

(d) the presence or absence of the reporter gene product in the presence of the individual substance or a mixture of a plurality of chemicals is compared with the presence or absence of the reporter gene product when this substance or mixture is absent, and,

(e) if appropriate, the compound or compounds by which the functionality of the splicing process is affected is(are) identified.

A preferred method of identifying splicing inhibitors is one which is characterized in that

(a) a DNA construct is generated in which the reporter gene is linked to the intron in such a way that the generation of the reporter gene product is ensured when splicing is adversely affected or fully suppressed;

(b) the DNA construct of step (a) is introduced into a host cell or a host organism, preferably into Ustilago maydis;

(c) the host cell or the host organism of step (b) is brought into contact with an individual substance or a mixture of a plurality of chemicals,

(d) the presence of the reporter gene product in the presence of the individual substance or a mixture of a plurality of chemicals is compared with the presence of the reporter gene product when this substance or mixture is absent, and,

(e) if appropriate, the compound or compounds by which the functionality of the splicing process is affected is(are) identified.

The present invention also relates to methods of finding chemicals which act on the expression of components of the spliceosome or of the direct units required for assembly or of the auxiliary components which are relevant in each case in a manner which leads to modulation of the splicing process, preferably to inhibition.

The present invention therefore also relates to a method of identifying compounds which affect the expression of components of the spliceosome, which method is characterized in that

(a) a DNA construct according to the invention (see above) is generated;

(b) the DNA construct of step (a) is introduced into a host organism or a host cell according to the invention;

(c) the host cell or the host organism of step (b) is brought into contact with an individual substance or a mixture of a plurality of chemicals,

(d) the presence or absence of the reporter gene product in the presence of the individual substance or a mixture of a plurality of chemicals is compared with the presence or absence of the reporter gene product when this substance or mixture is absent,

(e) the polypeptide and/or RNA composition of the spliceosome is determined, and,

(f) if appropriate, the compound or compounds by which the functionality of the splicing process is affected is/are identified.

dermatophytes such as, for example, Trichophyton spec., Microsporum spec., Epidermophyton floccosum or Keratomyces ajelloi which cause, for example, athlete's foot (tinea pedis), yeasts such as, for example, Candida albicans which cause, for example, candidal oesophagitis and dermatitis, Candida glabrata, Candida krusei or Cryptococcus neoformans, which may cause, for example, pulmonal cryptococcosis and torulosis,

moulds such as, for example, Aspergillus fumigatus, A. flavus, A. niger which cause, for example, bronchopulmonal aspergillosis or mycethemias, Mucor spec., Absidia spec. or Rhizopus spec., which cause, for example, zygomycoses (intravasal mycoses), Rhinosporidium seeberi which causes, for example, chronic granulomatous pharyngitis and tracheitis, Madurella myzetomatis which causes, for example, subcutaneous mycetomas, Histoplasma capsulatum which causes, for example, histoplasmosis and Darling's disease, Coccidioides immitis which causes, for example, pulmonal coccidioidomycosis and sepsis, Paracoccidioides brasiliensis which causes, for example, South American blastomycosis, Blastomyces dermatitidis which causes, for example, Gilchrist's disease and North American blastomycosis, Loboa loboi which causes, for example, keloidal blastomycosis and Lobo's disease, and Sporothrix schenckii which causes, for example, sporotrichosis (granulomatous dermatomycosis).

Modulators can be antagonists or inhibitors. Those of particular interest are, in the case of splicing, inhibitors which, owing to the elimination of the splicing process, lead to the corresponding action of abovementioned possible active substances.

The present invention therefore also relates in particular to the use of the spliceosome as target for active substances and to its use in methods of finding splicing modulators.

The present invention therefore also relates to the use of DNA constructs which indicate the splicing activity directly or indirectly, and of host cells or host organisms containing them for finding splicing modulators.

The term “modulator” as used in the present context is the generic term of agonist and antagonist, or activator and inhibitor. In this context, the term “agonist” or “activator” refers to a molecule which accelerates or increases the splicing activity, while the term “antagonist” or “inhibitor” refers to a molecule which slows down or prevents the splicing activity.

Modulators can be small organochemical molecules, peptides or antibodies which bind to the spliceosome and/or one of its constituents itself, which affect the assembly of the spliceosome and/or of one of the components involved therein, or which act on the expression of components of the spliceosome or of the direct units required for the assembly or of the corresponding auxiliary components. Modulators can be all those small organochemical molecules, peptides or antibodies which affect the splicing process in terms of correct location and/or timing.

The modulators preferably take the form of small organochemical compounds.

By acting on the splicing process, the modulators are capable of modifying the cellular processes in a manner which leads to the nonpathogenicity and/or death of fungi treated therewith.

By acting on the splicing process, the modulators are capable of modifying the cellular processes in a manner which leads to the death of pests treated therewith.

By acting on the splicing process, the modulators are capable of modifying the cellular processes in a manner which leads to the death of weeds treated therewith.

By acting on the splicing process, the modulators are capable of modifying the cellular processes in a manner which leads to the death of tumours treated therewith.

The present invention therefore also relates to modulators, preferably splicing inhibitors, which have been found with the aid of one of the methods described hereinabove or hereinbelow for identifying splicing modulators.

It has been unknown as yet that the splicing process in phytopathogenic fungi constitutes an outstanding target for fungicides and that compounds can be found, with the aid of the splicing process, which may be employed as fungicides. This possibility is described and demonstrated for the first time in the present application. Also provided are the auxiliaries required for demonstrating the functionality of the splicing process, such as DNA constructs and methods for their preparation.

The invention therefore relates to the use of splicing modulators as fungicides and/or antimycotics.

It has also been unknown as yet that the splicing process in animal pests constitutes an outstanding target for insecticides and that compounds can be found, with the aid of the splicing process, which may be employed as insecticides. This possibility is described and demonstrated for the first time in the present application. Also provided are the auxiliaries required for demonstrating the functionality of the splicing process, such as DNA constructs and methods for their preparation.

The present invention therefore furthermore relates to the use of splicing modulators as insecticides.

It has also been unknown as yet that the splicing process in weeds constitutes an outstanding target for herbicides and that compounds can be found, with the aid of the splicing process, which may be employed as herbicides. This possibility is described and demonstrated for the first time in the present application. Also provided are the auxiliaries required for demonstrating the functionality of the splicing process, such as DNA constructs and methods for their preparation.

The invention furthermore relates to the use of splicing modulators as herbicides.

The present invention furthermore extends to methods of finding chemicals which modify the expression of components of the spliceosome or of components which are required for the assembly of the spliceosome. Such “expression modulators” too can be new fungicidal active substances. Expression modulators can be small organochemical molecules, peptides or antibodies which bind to the regulatory regions of the nucleic acids encoding the polypeptides according to the invention. Moreover, expression modulators can be small organochemical molecules, peptides or antibodies which bind to a molecule which, in turn, binds to regulatory regions of the nucleic acids encoding the components of the spliceosome or components which are required for the assembly of the spliceosome, thus affecting their expression. Expression modulators may also be antisense molecules.

The present invention likewise relates to splicing expression modulators which are found with the aid of a method described hereinbelow for identifying expression modulators.

The invention also relates to the use of expression modulators as fungicides and/or antimycotics.

The invention also relates to the use of expression modulators as insecticides.

The invention also relates to the use of expression modulators as herbicides.

The invention also relates to the use of expression modulators as antitumour agents.

The methods according to the invention include HTS and UHTS. Both host cells and host organisms containing the DNA constructs according to the invention may be used for this purpose.

To find modulators of the polypeptides according to the invention, host cells or host organisms containing a DNA construct according to the invention can be incubated together with an individual substance or a mixture of a plurality of substances, each of which is a suitable candidate active substance. The ability of a candidate active substance of inhibiting the splicing activity is indicated by the reporter gene used in the DNA construct in such a way that either the gene product itself or the activity of the gene product shows a measurable effect.

For example, mixtures of potential candidate active substances may consist of 2, 10, 50, 100 or 1000 different compounds. However, any other mixtures are also possible.

If mixtures of candidate active substances are used in the method of finding splicing modulators, a positive result must be followed by a deconvolution, i.e. the actual active compound must be identified from the mixture. This is carried out for example by dividing the original mixture into mixtures with fewer compounds or into individual substances and repeating the process correspondingly.

The invention also relates to a method of finding splicing modulators, which is characterized in that a host cell or a host organism containing the DNA construct according to the invention is brought into contact with an individual substance or a mixture of substances, all of which are possible modulators, and the gene product or the activity of the reporter gene is detected or, if appropriate, quantified.

In general, a procedure is followed in which the test substances which are possible are dissolved in a suitable solvent (for example dimethyl sulphoxide, water or mixtures of both). A defined quantity/number of host cells or host organisms is added to this solution, and the gene product or the activity of the reporter gene is determined after specified periods.

In a preferred embodiment, the test substances are dissolved in dimethyl sulphoxide and a 5 μl aliquot of 100 μM solution is placed into an incubation vessel. Thereafter, 45 μl of cells (preferably U. maydis) containing the above-described DNA construct with the egfp gene as reporter gene and which had previously grown in a minimal medium up to an OD₆₀₀ of 1.25 are added. The fluorescence is measured after 0 h, 3 h and 6 h. The differences Δ3 h-0 h and Δ6 h-3 h in comparison with the controls are a measure of the effect of a substance.

Controls which are employed are firstly those strains which, while containing the DNA construct according to the invention, have not been brought into contact with potential active ingredients and, secondly, those strains which contain a DNA construct according to the invention which is mutated in such a way that splicing is continuously suppressed. The reporter gene is therefore always active in the method.

In a preferred embodiment, the negative control used is the strain DS#873, which contains the modified lga2 intron. The positive control used is strain DS#877, which contains the 5′mut-lga2 intron. FIG. 6 shows the increase in the fluorescence versus time in the two controls over 8 hours.

The method according to the invention for finding modulators of the splicing process may also be used in HTS and UHTS. To this end, the host cells/organisms are incubated together with the test substances and the fluorescence is measured for example directly in microtiter plates (MTP).

Using the abovementioned preferred U. maydis strains, and using an MTP, preferably one with 96 or 384 positions, especially preferably one with 384 positions, the mean relative fluorescence readings shown in the table hereinbelow are obtained.

| | Positive control | Negative control |
|---|---|---|
| Relative fluorescence | 22130 | 3408 |
| Standard deviation (%) | 574 (2.6) | 175 (5.1) |

The relative fluorescence reading of the positive control, i.e. simultaneously the maximum value of 100% prevented splicing, showed a mean which exceeded the corresponding value of the negative control by a factor of 6.5.

The present invention therefore also relates to a method of finding splicing modulators in an HTS or UHTS.

### EXAMPLES

Molecular-Biological Standard Methods

Molecular-biological standard methods (such as, for example, PCR, ligation, restriction, transformation of E. coli, RT-PCR, RNA isolation, Southern analysis, gel electrophoresis, DNA extraction from gels, plasmid preparation) are carried out as described by Sambrook et al. (1989).

Construction of the DNA Constructs

a) In a first step,

i) intron No. 1 of the lga2 gene is amplified from the genomic DNA of the U. maydis strain Um518 with the aid of the primers lga25′ (SEQ ID NO. 1) and CA52 (SEQ ID NO. 2), giving rise to the lga2 intron as a 74 bp fragment, and

ii) the egfp gene as the reporter gene is amplified from plasmid pCA123 with the primers CA53 (SEQ ID NO. 3) and 3′GFP-Not (SEQ ID NO. 4), giving rise to the egfp gene as a 726 bp fragment.

b) In a second step, the two amplificates of step (a) are cloned separately of one another into plasmid pCRIITopo (Invitrogen). The third nucleotide of the 5′ splicing site in the lga2 intron is mutated from G to A. After this step, firstly the plasmid pCRIITopo-lga2, which contains the intron sequence, and, secondly, the plasmid pCRIITopo-UeGFP, which contains the reporter gene, are obtained.

c) In a third step,

i) the lga2 intron is excised from the plasmid pCRIITopo-lga2 as a 78 bp BglII/SphI fragment,

ii) the egfp gene is excised from plasmid pCRIITopo-UeGFP as a 726 bp SphI/NotI fragment,

iii) plasmid pCA123 is restricted with the restriction enzymes BamHI and NotI, and

iv) the three resulting fragments are ligated together, giving rise to plasmid p123-lga2-eGFP.

To obtain the DNA construct which is mutated at the 5′-splicing site of the intron sequence in such a way that splicing can no longer take place, the primer lga25′mut (SEQ ID NO. 5) is used in step (a), finally giving rise to plasmid p123-lga25′mut-eGFP.

Construction of Plasmid pCA123

The otef promoter is isolated from plasmid pOTEF-SG (Spellig et al. (1996)) as an 890 bp PvuII/NcoI fragment and ligated into the PvuII/NcoI-cut vector pTEF-SG (Spellig et al. (1996)). In the resulting plasmid, the SGFP gene is excised by restriction with NcoI/NotI and replaced by the NcoI/NotI-cut EGFP allele from pEGFP-N1 (Clontech). The resulting plasmid is named pCA123. It consists of a pSP72 backbone, the otef promoter, the eGFP gene (Clontech) and the cbx resistance cassette.

Introduction of the DNA Constructs Into U. mavdis Cells

The DNA constructs are introduced by linearizing plasmid p123-lga2-eGFP with the restriction enzyme SspI and transforming it into the genome of the haploid U. maydis strain Um518 by the PEG/protoplast method (cf. Schulz et al. (1990)). Cleavage with SspI causes integration of the constructs preferentially at the cbx locus of U. maydis. The same procedure is followed with plasmid p123-lga25′mut-eGFP.

To determine the site of integration of the splicing constructs, genomic DNA is first isolated from U. maydis (cf. Hoffmann and Winston (1987)) and subsequently digested with the restriction enzymes HindIII and BamHI. For the detection, a 283 bp PCR fragment from plasmid pCBX122, which contains the cbx resistance cassette, is used as probe in the case of integration into the cbx locus of Ustilago maydis (Keon et al. (1991)). Detection is performed using the Dig system (Roche). Amplification was performed by PCR using the primers CBX-S3 (SEQ ID NO. 12) and CBX-A4 (SEQ ID NO. 13).

The fact that the constructs are always integrated at the same gene locus makes possible a comparable expression in the different strains. The individual integration events are confirmed by means of PCR and by Southern blot analysis (see FIG. 4). Strains DS#873 and DS#877 bear in each case two copies of the constructs p123-lga2-eGFP and p123-lga25′mut-eGFP.

Detection of GFP Expression

Analysis of GFP Fluorescence

The U. maydis reporter strains are incubated at 28° C. in PD medium (potato dextrose) to an optical density OD₆₀₀ of 0.8, harvested by centrifugation (2200 g, Heraeus) and brought to the OD₆₀₀ stated in each case with minimal medium (Holliday (1974)) in 0.1% Kelzan (Monsanto). The cells are subsequently transferred into 384-well MTPs (Greiner, black) using a Multidrop device (Labsystems). Measurement was effected in a Tecan ultra-fluorescence reader (Tecan) (excitation wavelength 480 nm, bandwidth 10 nm; emission wavelength 510 nm, bandwidth 10 nm; gain factor 50; 3 flashes).

Detection of eGFP Expression by RT-PCR

For RT-PCR experiments, total RNA is isolated from U. maydis liquid cultures (Schmitt et al. (1990)). Then, polyA⁺RNA is prepared with the aid of magnetic Poly-dT beads following the manufacture's (Dynal) instructions. 0.1-5 ng of polyA⁺ RNA are employed per RT-PCR experiment, and the “SYBR green II” kit (Roche) is employed for the amplification and the fluorescent labelling. A light cycler PCR machine (Roche) is employed for the amplification. The RT-PCR was carried out following the manufacturer's (Roche) instructions. The following primer combinations were used: GFP5′ (SEQ ID NO. 6)/GFP3′ (SEQ ID NO. 7); 5′UTR (SEQ ID NO. 8)/GFP3′ (SEQ ID NO. 7) and intron (SEQ ID NO. 9)/GFP3′ (SEQ ID NO. 7). The amplicons were separated in a 1% agarose gel.

Adaptation of the Test Strains to 384-Well MTP Format

Test strains for identifying splicing-inhibitory substances are incubated in PD medium and harvested at an OD₆₀₀ of 0.8, washed in water and subsequently taken up in minimal medium in such a way that the OD₆₀₀ is 2.5. 50 μl portions of the culture are diluted 1:1 in minimal medium with 0.2% Kelzan (Monsanto) so that an OD₆₀₀ of 1.25 is obtained. 50 μl portions of the cultures are subsequently pipetted into the cavities of MT plates. The fluorescence is determined as described above.

To analyse the splicing test strains for increase in the GFP fluorescence as a function of time, the GFP fluorescence kinetics of the strains are determined over a period of 8 hours. To this end, the strains are employed in an OD600 of 1.5 (fluorescence measurement as above).

Inhibition Assay for Identifying Splicing-Inhibitory Substances

The test substances are dissolved in DMSO and diluted in water (final concentration 100 μm). 5 μl portions of this solution are introduced into a 384-well MTP. 45 μl of U. maydis cells with an OD₆₀₀ of 1.25 in minimal medium with 0.1% Kelzan (Monsanto) are subsequently added. The fluorescence is then determined in a fluorimeter. Further measurements are carried out after 3 h and 6 h. The limit for GFP induction is set at 1.5×the mean of the background fluorescence.

Information on the Sequence Listing

SEQ ID NO. 1: DNA sequence of the primer lga 25′

SEQ ID NO. 2: DNA sequence of the primer CA52

SEQ ID NO. 3: DNA sequence of the primer CA53

SEQ ID NO. 4: DNA sequence of the primer 3′-GFP-Not

SEQ ID NO. 5: DNA sequence of the primer lga 25′mut

SEQ ID NO. 6: DNA sequence of the primer GFP5′

SEQ ID NO. 7: DNA sequence of the primer GFP3′

SEQ ID NO. 8: DNA sequence of the primer 5′UTR

SEQ ID NO. 9: DNA sequence of the primer intron

SEQ ID NO. 10: DNA sequence of the modified intron No. 1 from the Ustilago maydis lga2 gene (functional)

SEQ ID NO. 11: DNA sequence of the mutated intron No. 1 from the Ustilago maydis lga2 gene (functionless)

SEQ ID NO. 12: DNA sequence of the primer CBX-S3

SEQ ID NO. 13: DNA sequence of the primer CBX-A4

SEQ ID NO. 14: DNA sequence of the oma promoter

### INFORMATION ON THE FIGURES

FIG. 1: Schematic representation of the two transesterification steps of the splicing process

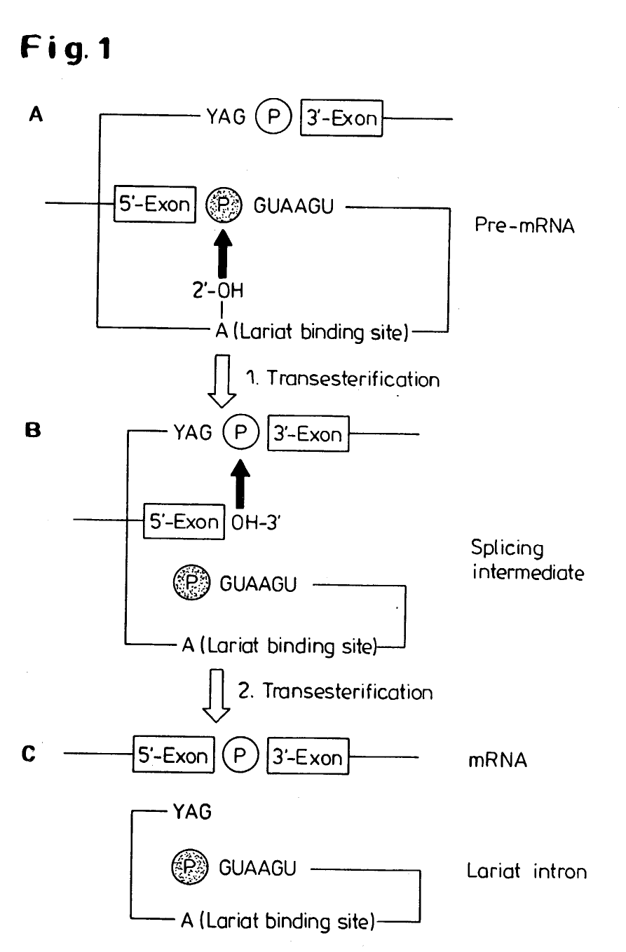

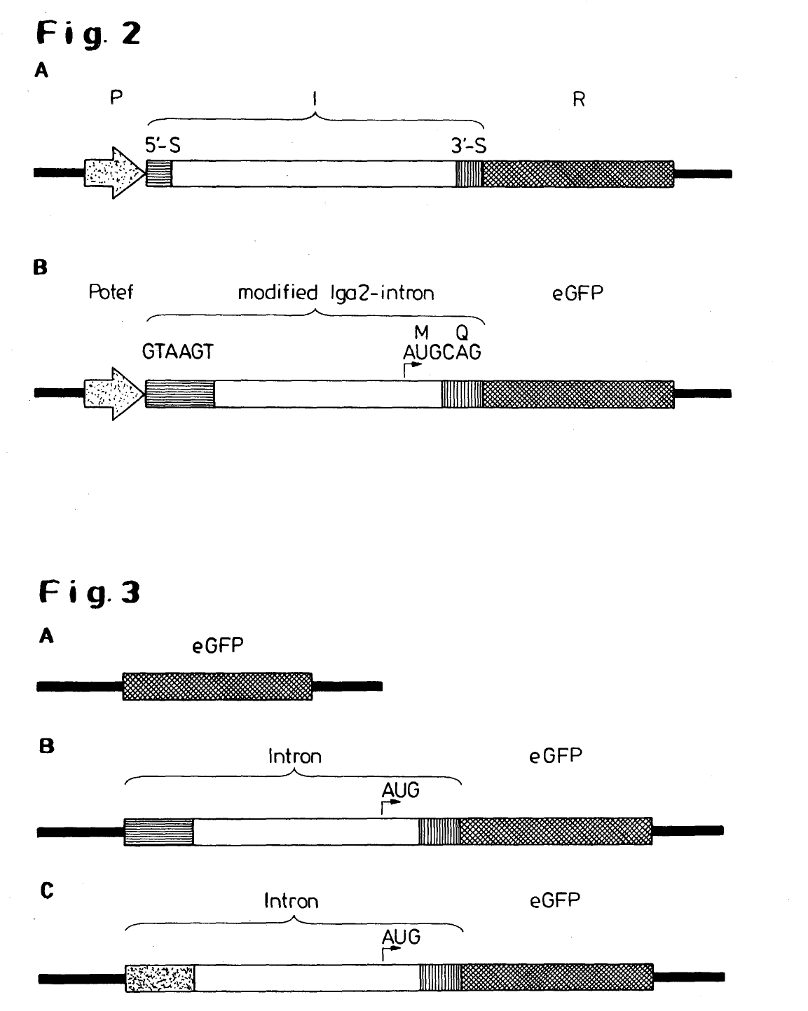

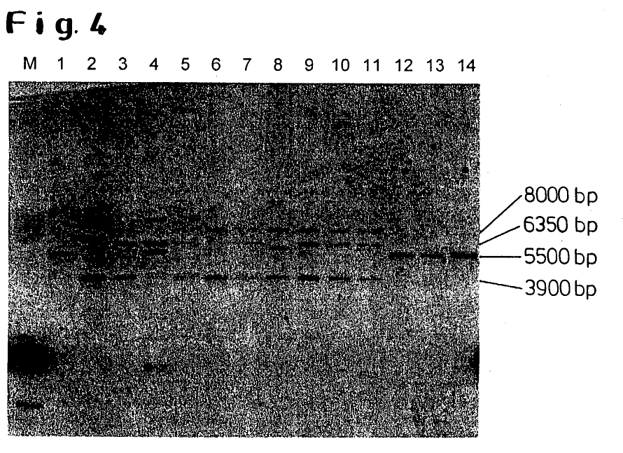

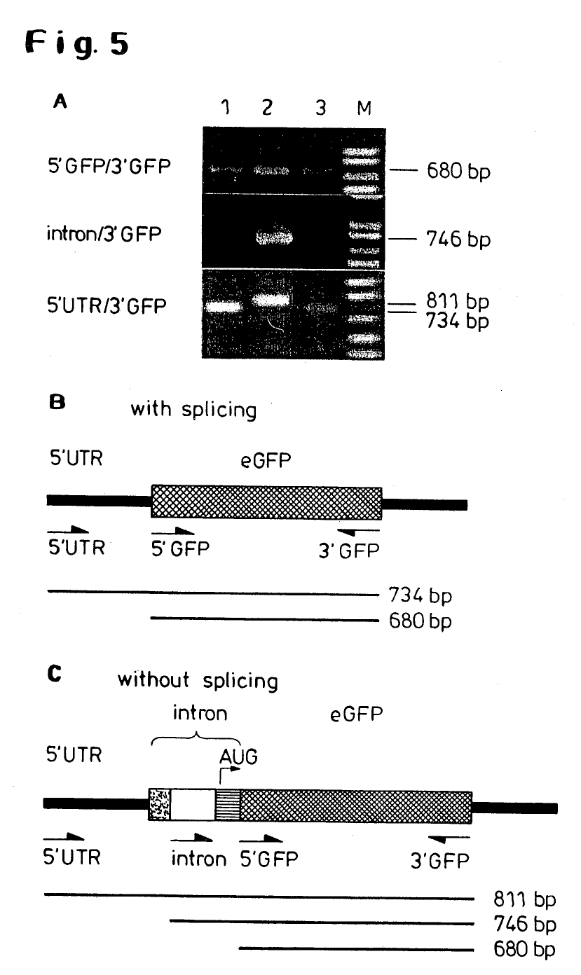

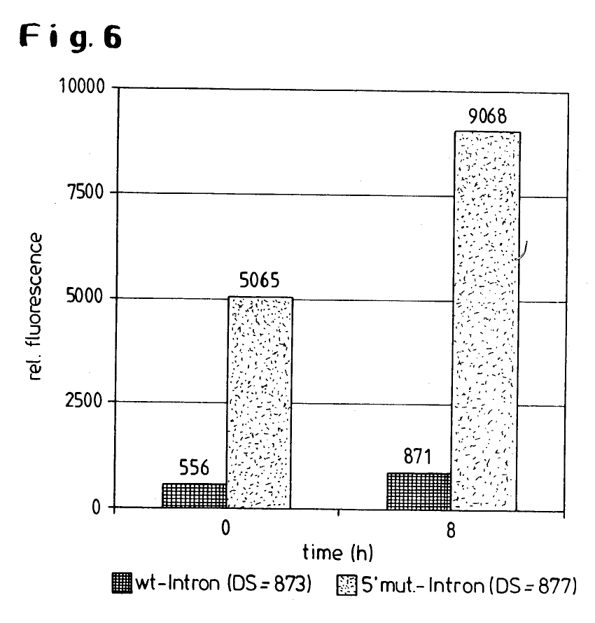

A: Transesterification step 1:

Within the pre-mRNA, the 2′-OH group of the invariant adenosine unit of the Lariat binding site carries out a nucleophilic attack on the 5′-phosphate group of the guanosine unit of the intron, giving rise to what is known as the Lariat structure.

B: Transesterification step 2:

In the second step, the free 3′-OH group of the 5′ exon eliminated during the first reaction attacks the phosphodiester bond at the 3′-splicing site.

C: Products:

The result of the two transesterifications of steps A and B are the mRNA and the Lariat intron.

In the figures, A represents adenosine, G guanosine, C cytosine, U uracil. Y represents T or C. The phosphate group which takes part in the first transesterification is shown by an encircled “P” against a white background, while the phosphate group participating in the second esterification is shown by an encircled “P” against a grey background. The 5′- and 3′-splicing consensus sequences of higher eukaryotes are shown.

FIG. 2: Schematic representation of the DNA constructs

A: Example of the general construction of a DNA construct in whose presence in a eukaryotic cell the splicing activity can be studied. A DNA construct according to the invention consists of a promoter (P), an intron sequence [(I) including the flanking 5′ splicing site (5′-S) and the 3′ splicing site (3′-S)] and the reporter gene (R).

B: Preferred construction of a DNA construct according to the invention consisting of the promoter Potef and the modified intron No. 1 from the U. maydis lga2 gene flanked by the 5′ splicing site with the sequence GTAAGT and the 3′ splicing site of the sequence CAG (encoding the amino acid glutamine (Q)). The start codon AUG (encoding the amino acid methionine (M)) is located upstream of the 3′ splicing site. The eGFP gene is used as the reporter gene.

In this figure, the promoters are shown in each case by a dotted arrow. The intron sequence is shown by a white bar, the 5′ splicing site being indicated by a horizontally hatched bar and the 3′ splicing site by a vertically hatched bar. The reporter gene is shown by a black and white chequered bar.

FIG. 3: Representation of the mRNAs in the individual splicing reporter strains

A: Owing to splicing, the intron is no longer present in the mature mRNA. This also removed the start codon for the translation of GFP. Fluorescence is therefore not observed.

B: Splicing is prevented by an inhibitor. The mRNA contains the start codon for the translation GFP, whereby fluorescence is observed.

C: The intron sequence was mutated in the 5′ splicing site, whereby no splicing takes place even in the absence of an inhibitor. GFP is always expressed, and the maximum fluorescence can be observed. This construct acts as the positive control.

In this figure, GFP is shown by a black and white chequered bar. The intron sequences are shown by a white bar, the 5′ splicing site being indicated by a horizontally hatched bar and the 3′ splicing site by a vertically hatched bar. The mutated 5′ splicing site in Figure C is shown by a grey bar.

FIG. 4: Southern analysis of the splicing strains, integration of the constructs in the cbx locus of U. maydis.

In each case 2.5 μg of the genomic DNA in the case of transformants and 3 ng in the case of plasmids were cut with BamHI, separated by size, blotted and hybridized with a DNA probe which is specific for the cbx gene.

| | |
|---|---|
| Lane M: | 1 kb + size marker |
| Lane 1: | Um518 |
| Lane 2: | UMA3 |
| Lanes 3-7: | 5181ga2eGFP candidates |
| Lanes 8-11: | 5181ga2muteGFP candidates |
| Lane 12: | plga2eGFP |
| Lane 13: | plga2eGFP#2 |
| Lane 14: | plga2eGFPH#7 |

FIG. 5: Detection of the various GFP mRNA species in the splicing test strains by RT-PCR

A: RT-PCR detection of the GFP mRNA species during the various splicing states. The mRNA was isolated from U. maydis liquid cultures which had grown for 12 h in PD medium. Three primer combinations were used for the RT-PCR reaction (5′GFP/3′GFP, top third of the figure; intron/3′GFP, middle third of the figure; 5′UTR/3′GFP, bottom third of the figure).

Lane 1: Test strain GFP with wild-type intron (strain DS#873)

Lane 2: Test strain GFP with 5′mut intron (strain DS#877)

Lane 3: Test strain GFP control strain (strain UMA3)

Lane 4: 1 kb+ size marker

B: Schematic representation of the mRNA and of the PCR products in the case of active splicing (not to scale)

C: Schematic representation of the mRNA and PCR products in the case of inactive splicing (not to scale)

Analogously to FIG. 2 and FIG. 3, the mRNA in FIG. 5B and FIG. 5C is shown as a bar diagram. The colours and patterns are the same as in FIG. 3.

An arrow under each of the bar diagrams of the mRNA indicates the primers and their respective attachment position. The primers used are 5′UTR, 5′GFP, 3′GFP and intron. The lengths of the RT-PCR products to be expected are shown as black bars, with the length being stated in bp (base pairs).

FIG. 6: Increase in the GFP fluorescence in the splicing test strains as a function of time.

The relative fluorescence of the test strain is plotted versus time.

### REFERENCES

Berget S M, Moore C, Sharp P A (1977): Spliced segments at the 5′ terminus of adenovirus 2 late mRNA. Proc. Natl. Acad. Sci. USA 74, 3171-3175.

Bölker M, Urban M, Kahmann R (1992): The α mating type locus of U. maydis specifies cell signaling components. Cell 68, 441-450.

Bottin A, Kämper J, Kahmann R (1995): Isolation of a carbon source regulated gene from Ustilago maydis. Mol. Gen. Genet. 253, 342-452.

Fischer U, Liu Q, Dreyfuss G (1997): The SMN-SIP1 complex has an essential role in spliceosomal snRNP biogenesis. Cell 90, 1023-1029.

Grabowski P J, Seiler S R, Sharp P A (1985): A Multicomponent Complex Is Involved in the Splicing of Messenger RNA Precursors. Cell 42, 345-353.

Holden D W, Kronstad J W, Leong S A (1989): Mutation in a heat-regulated hsp70 gene of Ustilago maydis. EMBO J 8, 1927-1934.

Holliday R (1974): Ustilago maydis. In King R C (ed), Handbook of Genetics. Vol. 1, Plenum, N.Y., pp.575-595.

Hoffmann C S, Winston F (1987): A ten-minute DNA preparation from yeast efficiently releases autonomous plasmids for transformation in E. coli. Gene. Gene 267-272.

Keon J P R, White G A, Hargraves J A (1991): Isolation, characterization and sequence of a gene conferring resistance to the systemic fungicide carboxin from the maize smut pathogen Ustilago maydis. Curr. Genet. 19, 475-481.

Sambrook J (1977): Adenovirus amazes at Cold Spring Harbor. Nature 268, 101-104.

Sambrook J, Fritsch E F, Maniatis T (1989): Molecular cloning: A laboratory manual. Cold Spring Harbor Laboratory Press, Cold Spring Harbor, N.Y.

Schmitt B E, Brown T A, Trumpower B L (1990): A rapid and simple method for preparation of RNA from Saccharomyces cerevisiae. Nucleic Acids Res. 18, 3091-3092.

Schulz B, Banuett F, Dahl M, Schlesinger R, Schäfer W, Martin T, Herskowitz I, Kahmann R (1990): The b Alleles of Ustilago maydis, Whose Combinations Program Pathogenic Development, Code for Polypeptides Containing a Homeo-domain-related Motif. Cell 60, 295-306.

Sherman L, Sleeman J, Dall P, Hekele A, Moll J, Ponta H, Herrlich P (1996): The CD44 proteins in embryonic development and in cancer. Curr. Top. Microbiol. Immunol. 213, 249-269.

Spellig T, Bottin A, Kahmann R (1996): Green fluorescent protein (GFP) as a new vital marker in the phytopathogenic fungus Ustilago maydis. Mol. Gen. Genet. 252, 503-509.

Urban M, Kahmann R, Bölker M (1996): The biallelic α mating type locus of Ustilago maydis: remnants of an additional pheoromone gene indicate evolution from a multiallelic ancestor. Mol. Gen. Genet. 250, 414-420.

Zanelli E, Henry M, Charvet B, Malthiery Y (1990): Evidence for an alternate splicing in the thyroperoxidase messenger from patients with Graves' disease. Biochem. Biophys. Res. Commun. 170, 735-741.

1. DNA construct comprising
a) a promoter which is active in eukaryotes,
b) a DNA sequence which has all elements of a functional intron, and
c) a reporter gene,
all three elements being functionally linked to each other.

2. DNA construct according to claim 1, characterized in that the reporter gene is linked to the intron such that the formation of the reporter gene product is ensured in the case of correct splicing.

3. DNA construct according to claim 1, characterized in that the reporter gene is linked to the intron such that the formation of the reporter gene product is ensured in the case of adversely affected or fully suppressed splicing.

4. DNA construct according to one of claims 1 to 3 characterized in that the promoter is derived from Ustilago maydis.

5. DNA construct according to claim 4, characterized in that the promoter is selected from one of the following promoters:
a) (regulable) crg1 promoter,
b) (constitutive) hsp70 promoter,
c) synthetic otef promotor,
d) synthetic oma promoter with the sequence shown in SEQ ID NO. 14.

6. DNA construct according to one of claims 1 to 5, characterized in that the intron sequence is recognized as intron by the spliceosome and, in the case of intact splicing, is excised from the DNA.

7. DNA construct according to claim 6, characterized in that the 5′ splicing site of the intron sequence comprises the nucleotide 5′-GU-3′.

8. DNA construct according to one of claims 6 or 7, characterized in that the 5′ splicing site comprises the nucleotides 5′-GUAAGU-3′.

9. DNA construct according to claim 6, characterized in that the 3′ splicing site of the intron sequence comprises the nucleotides 5′-AG-3′.

10. DNA construct according to one of claims 6 or 9, characterized in that the 3′ splicing site comprises the nucleotides 5′-YAG-3′, where Y represents a pyrimidine base.

11. DNA construct according to one of claims 6 to 10, characterized in that the intron sequence contains no start and/or stop codons.

12. DNA construct according to one of claims 6 to 11, characterized in that the intron sequence is selected among one of the four introns of the lga2 gene and one of the three introns of the pra1 gene from Ustilago maydis.

13. DNA construct according to claim 12, characterized in that the intron has the sequence shown in SEQ ID NO. 10.

14. DNA construct according to one of claims 1 to 13, characterized in that it comprises a reporter gene among the following group of reporter genes: GFP and its variants and its derivatives (for example eGFP, yGFP, cGFP), lacZ, LUX, GUS, CAT, orotidine 5′-monophosphate decarboxylate, nitrate reductase.

15. DNA construct according to claim 14, characterized in that the reporter gene is eGFP.

16. DNA construct according to one of claims 1 to 15, consisting of the otef promoter, an intron with a sequence as shown in SEQ ID NO. 10 and the eGFP gene.

17. DNA construct according to one of claims 1 to 15 consisting of the oma promoter with a sequence as shown in SEQ ID NO. 14, the intron with a sequence as shown in SEQ ID NO. 10 and the eGFP gene.

18. A method of generating a DNA construct according to claim 1, characterized in that,
a) in a first step,
i) a suitable intron is amplified, the sequence of the intron optionally being modified specifically by selecting a suitable primer,
ii) a suitable reporter gene is amplified,
b) in a second step, the two amplificates of step (a) independently of one another are cloned into a suitable plasmid I, giving rise to the two plasmids II (intron) and III (reporter gene),
c) in a third step,
i) the intron fragment is excised from plasmid II (intron) using suitable restriction enzymes,
ii) the reporter gene fragment is excised from plasmid III (reporter gene) using suitable restriction enzymes,
iii) a suitable vector is restricted, and
iv) the three resulting fragments are ligated in such a manner that a plasmid V is obtained in which the intron sequence and the reporter gene are operably linked.

19. Host cells and host organisms, characterized in that they contain DNA constructs according to one of claims 1 to 17.

20. Host cells and host organisms according to claim 19, characterized in that they are eukaryotic cells, mammalian cell lines or fungal cell lines.

21. Host cells and host organisms according to one of claims 19 or 20, characterized in that they are fungal cells, insect cells, plant cells, frog oocyte cells or else Volvox spheroids, Drosophila embryos or Daphnia larvae.

22. Host cells and host organisms according to claim 21, characterized in that they are fungal cells.

23. Host cells and host organisms according to claim 22, characterized in that they are cells of Saccharomyces cerevisiae, Magnaporthe grisea, Aspergillus nidulans, Cochliobulus heterostrophus, Nectria hematococca, Botrytis cinerea, Gaeumannomyces sp., Pichia pastoris and Ustilago maydis.

24. Host cells and host organisms according to claim 23, characterized in that they are Ustilago maydis.

25. Method for detecting the functionality of the splicing process in vivo.

26. Method according to claim 25, characterized in that
(A) a DNA construct according to one of claims 1 to 17 is generated,
(B) this DNA construct is introduced into a host cell or a host organism, and
(C) the presence or absence of the reporter gene product is verified.

27. Method according to claim 26, characterized in that
(A) a DNA construct according to claim 3 is generated,
(B) this DNA construct is introduced into a host cell or a host organism, and
(C) the presence of the reporter gene product is verified.

28. Method of identifying splicing inhibitors, characterized in that
(a) a DNA construct according to one of claims 1 to 17 is generated;
(b) the DNA construct of step (a) is introduced into a host cell or a host organism;
(c) the host cell or the host organism of step (b) is brought into contact with an individual substance or a mixture of a plurality of chemicals,
(d) the presence or absence of the reporter gene product in the presence of the individual substance or a mixture of a plurality of chemicals is compared with the presence or absence of the reporter gene product when this substance or mixture is absent, and
(e) if appropriate, the compound or compounds by which the functionality of the splicing process is affected is or are identified.

29. Method according to claim 28, characterized in that
(a) a DNA construct according to claim 3 is generated;
(b) the DNA construct of step (a) is introduced into a host cell or a host organism;
(c) this host cell or the host organism of step (b) is brought into contact with an individual substance or a mixture of a plurality of chemicals,
(d) the presence of the reporter gene product in the presence of the individual substance or a mixture of a plurality of chemicals is compared with the presence of the reporter gene product when this substance or mixture is absent, and
(e) if appropriate, the compound or compounds by which the functionality of the splicing process is affected is or are identified.

30. Method of identifying compounds which affect the expression of components of the spliceosome, characterized in that
(a) a DNA construct according to one of claims 1 to 17 is generated;
(b) the DNA construct of step (a) is introduced into a host cell or a host organism;
(c) the host cell or the host organism of step (b) is brought into contact with an individual substance or a mixture of a plurality of chemicals,
(d) the presence or absence of the reporter gene product in the presence of the individual substance or a mixture of a plurality of chemicals is compared with the presence or absence of the reporter gene product when this substance or mixture is absent,
(e) the polypeptide and/or RNA composition of the spliceosome is determined, and
(f) if appropriate, the compound or compounds by which the functionality of the splicing process is affected is or are identified.

31. Method according to one of claims 26 to 30, characterized in that host cells or host organisms according to one of claims 19 to 24 are used.

32. Use of a DNA construct according to one of claims 1 to 17 in the method according to one of claims 26 to 31.

33. Use of the splicing process in methods of identifying fungicidal, insecticidal and/or herbicidal compounds.

34. Use of splicing modulators as fungicides or antimycotics.

35. Use of splicing modulators as insecticides or herbicides.

36. Splicing modulators found with the aid of a method according to one of claims 26 to 31.


SEQUENCE LISTING (14 sequences)

SEQ ID NO: 1 - DNA, 28 bp, Artificial Sequence
  DNA sequence for primer lga25′
gccagatctaggtaagttgcttcaaatc

SEQ ID NO: 2 - DNA, 26 bp, Artificial Sequence
  DNA sequence for primer CA52
ctgcatgcgaaaatgaaaagtcgacg

SEQ ID NO: 3 - DNA, 28 bp, Artificial Sequence
  DNA sequence for primer CA53
tagcatgcaggtgagcaagggcgaggag

SEQ ID NO: 4 - DNA, 25 bp, Artificial Sequence
  DNA sequence for primer 3′-GFP-Not
agcggccgcttacttgtacagctcg

SEQ ID NO: 5 - DNA, 28 bp, Artificial Sequence
  DNA sequence for primer lga25′mut
gccagatctacttaagttgcttcaaatc

SEQ ID NO: 6 - DNA, 18 bp, Artificial Sequence
  DNA sequence for primer GFP5′
gtgagcaagggcgaggag

SEQ ID NO: 7 - DNA, 21 bp, Artificial Sequence
  DNA sequence for primer GFP3′
ctagattacttgtacagctcg

SEQ ID NO: 8 - DNA, 20 bp, Artificial Sequence
  DNA sequence for primer 5′UTR
cacagacaacatcatccacg

SEQ ID NO: 9 - DNA, 21 bp, Artificial Sequence
  DNA sequence for primer intron
tgcttcaaatcagattacact

SEQ ID NO: 10 - DNA, 78 bp, Artificial Sequence
  DNA sequence of the modified intron No. 1 from the Ustilago maydis lga2 gene (functional)
gatctaggtaagttgcttcaaatcagattacactggataagaacatatctgacgtcgacttttcattttcgcatgcag

SEQ ID NO: 11 - DNA, 78 bp, Artificial Sequence
  DNA sequence of the mutated intron No. 1 from the Ustilago maydis lga2 gene (functionless)
gatctacttaagttgcttcaaatcagattacactggataagaacatatctgacgtcgacttttcattttcgcatgcag

SEQ ID NO: 12 - DNA, 23 bp, Artificial Sequence
  DNA sequence for primer CBX-S3
agtcgtacacctggacctcaacc

SEQ ID NO: 13 - DNA, 21 bp, Artificial Sequence
  DNA sequence for primer CBX-A4
ggctcgatggatcggtactgc

SEQ ID NO: 14 - DNA, 1338 bp, Artificial Sequence
  DNA sequence of the oma promoter
tcgagtgccacacttgtcacaatacgcaggaaccgccgttcgcacactatacgttggtgtggtcttgcaaatatgcacaccgtccatcaagcttatcgataccgtcgagtgccacacttgtcacaatacgcaggaaccgccgttcgcacactatacgttggtgtggtcttgcaaatatgcacaccgtccatcaagcttatcgataccgtcgagtgccacacttgtcacaatacgcaggaaccgccgttcgcacactatacgttggtgtggtcttgcaaatatgcacaccgtccatcaagcttatcgataccgtcgaggtcgagtgccacacttgtcacaatacgcaggaaccgccgttcgcacactatacgttggtgtggtcttgcaaatatgcacaccgtccatcaagcttatcgataccgtcgagtgccacacttgtcacaatacgcaggaaccgccgttcgcacactatacgttggtgtggtcttgcaaatatgcacaccgtccatcaagcttatcgataccgtcgagtgccacacttgtcacaatacgcaggaaccgccgttcgcacactatacgttggtgtggtcttgcaaatatgcacaccgtccatcaagcttatcgataccgtcgaggtcgagtgccacacttgtcacaatacgcaggaaccgccgttcgcacactatacgttggtgtggtcttgcaaatatgcacaccgtccatcaagcttatcgataccgtcgagtgccacacttgtcacaatacgcaggaaccgccgttcgcacactatacgttggtgtggtcttgcaaatatgcacaccgtccatcaagcttatcgataccgtcgagtgccacacttgtcacaatacgcaggaaccgccgttcgcacactatacgttggtgtggtcttgcaaatatgcacaccgtccatcaagcttatcgataccgtcgaggtcgacggtatcgataagcttgatatcgaattgatcccggtcaccttcctggatgagaagaccaacttcgattactatgtctgcgcagggaaaggtgtaactgctggctgctcagtgtacgattgtcgaagaagcatctcgggatgtcagcactcttactcacctggtgcgttgcgctcatgagcccttgagacaagcgaagtccatcttctgcaacgcaatgctcgacatcactgagacggtaccgtcaaggatataagggagcaattggatatcaatccgacagccaaacctcatccactctcactttcacactctaacttatacgatcacttctcgcccgttcttttgaacatcaaatcaactaccttactctatcaggatc